(19) United States
(12) Patent Application Publication
(10) Pub. No.: US 2024/0188895 A1
(43) Pub. Date: Jun. 13, 2024

(54) MODEL TRAINING METHOD, SIGNAL RECOGNITION METHOD, APPARATUS, COMPUTING AND PROCESSING DEVICE, COMPUTER PROGRAM, AND COMPUTER-READABLE MEDIUM

(71) Applicant: BOE Technology Group Co., Ltd., Beijing (CN)

(72) Inventors: Chunhui Zhang, Beijing (CN); Zhenzhong Zhang, Beijing (CN)

(73) Assignee: BOE Technology Group Co., Ltd., Beijing (CN)

(21) Appl. No.: 17/772,405

(22) PCT Filed: Jul. 27, 2021
(86) PCT No.: PCT/CN2021/108605

§ 371 Date: Apr. 27, 2022

(51) Int. Cl. A61B 5/00

(52) U.S. Cl. CPC A61B 5/7267

(57) ABSTRACT

Model training method, signal recognition method, apparatus, computing and processing device, computer program, and computer-readable medium. The model training method comprises: acquiring a training sample set, training sample set includes sample electrocardio-signals and abnormal labels of sample electrocardio-signals, and abnormal labels include a target abnormal labels and at least one related abnormal labels; inputting sample electrocardio-signals into multi-task model, training multi-task model based on a multi-task learning mechanism according to an output of multi-task model and the abnormal labels; multi-task model includes a target task model and at least one related task model, a target output of the target task model is target abnormality labels of inputted sample electrocardio-signals, and a target output of related task model is related abnormal labels of inputted sample electrocardio-signals; determining target task model after trained as target-abnormality-recognition model, and target-abnormality-recognition model is configured for recognizing target abnormality in the electrocardio-signals inputted into target-abnormality-recognition model.

### TECHNICAL FIELD

The present disclosure relates to the technology field of computer, and more particularly, relates to a model training method, a signal recognition method, an apparatus, a computing and processing device, a computer program, and a computer-readable medium.

### BACKGROUND

The Electrocardiogram (ECG) is one of the effective methods of clinical diagnosis for cardiovascular diseases. In recent years, the classification and identification of abnormal electrocardio-signals have been extensive researched and gained attention.

The classification and recognition method based on the deep learning has the advantage of automatically extracting features, but the deep learning generally has a plurality of hidden layers, deep network structure, and contains a large number of the parameters that need to be trained. A lot of training data are needed to train an optimum model. Training a multi-classification model, each type of electrocardio abnormality requires a large amount and balanced training data in order to achieve a better classification effect.

### SUMMARY

The present disclosure provides a model training method, comprising:
acquiring a training sample set, wherein the training sample set includes sample electrocardio-signals and abnormal labels of the sample electrocardio-signals, and the abnormal labels include target abnormal labels and at least one related abnormal label;
inputting the sample electrocardio-signals into a multi-task model, and training the multi-task model based on a multi-task learning mechanism according to an output of the multi-task model and the abnormal labels; wherein, the multi-task model includes a target task model and at least one related task model, a target output of the target task model is target abnormality labels of the inputted sample electrocardio-signals, and a target output of the related task model is the related abnormal labels of the inputted sample electrocardio-signals; and
determining the target task model after trained as a target-abnormality-recognition model, wherein the target-abnormality-recognition model is configured for recognizing a target abnormality in the electrocardio-signals inputted into the target-abnormality-recognition model.

In an optional embodiment, the step of training the multi-task model based on the multi-task learning mechanism comprises: adjusting the parameters of each of the related task models, and adjusting the parameters of the target task model according to the parameters of the at least one related task model.

In an optional embodiment, the step of adjusting the parameters of the target task model according to the parameters of the at least one related task model comprises:
determining a regularized-loss item according to the parameters of the target task model and the parameters of the at least one related task model, and the regularized-loss item is configured to make the parameters of the target task model and the parameters of the at least one related task model similar; and
determining a first loss value according to the regularized-loss item, and adjusting the parameters of the target task model with a goal of minimizing the first loss value.

In an optional embodiment, the step of determining the regularized-loss item according to the parameters of the target task model and the parameters of the at least one related task model comprises:
determining the regularized-loss item according to the following formula:

R(θ₁,θ₂ , . . . , θ_{M})=λ(|θ₁−θ₂|²+. . . +|θ₁−θ_{M}|²)

wherein, the R (θ₁,θ₂ , . . . , θ_{M}) refers to the regularized-loss item, the M refers to a total number of the target task model and the related task model in the multi-task model, the θ₁ refers to the parameters of the target task model, the θ₂ , . . . , θ_{M} respectively represents the parameters of each of the related task models, and the λ represents a preset parameter.

In an optional embodiment, the step of adjusting the parameters of each of the related task models comprises:
inputting the sample electrocardio-signals into a first related task model, and inputting the output of the first related task model and first related abnormal labels into a preset second loss function, to obtain a second loss value, adjusting the parameters of the first related task model with a goal of minimizing the second loss value, wherein, the first related task model is any one of the at least one related task model, and the first related abnormal labels is any one of the at least one related abnormal labels;
before the step of determining the first loss value according to the regularized-loss item, the method further comprises:
inputting the sample electrocardio-signals into the target task model, inputting the output of the target task model and the target abnormal labels into a preset experience loss function, to obtain an experience loss item; and
the step of determining a first loss value according to the regularized-loss item comprising:
calculating the sum of the experience loss item and the regularized-loss item, to obtain the first loss value.

In an optional embodiment, the second loss value and the experience loss function are both cross-entropy loss functions.

In an optional embodiment, the target task model and the related task model share a common feature extraction layer, and the common feature extraction layer is configured to extract the common features of the target abnormality and a related abnormality, the step of adjusting the parameters of the target task model according to the parameters of the at least one related task model comprises:
sharing parameters of the common feature extraction layer in the at least one related task model as parameters of the common feature extraction layer in the target task model, and adjusting the parameters of the target task model after the parameters are shared.

In an optional embodiment, the step of adjusting the parameters of each of the related task models comprises:
inputting the sample electrocardio-signals into a second related task model, and inputting an output of the second related task model and the second related abnormal labels into a preset third loss function, to obtain a third loss value, and adjusting parameters of the second related task model with a goal of minimizing the third loss value, wherein the parameters of the second related task model include the parameters of the common feature extraction layer, wherein, the second related task model is any one of the at least one related task model, and the second related abnormal label is any one of the at least one related abnormal label; and
the step of adjusting the parameters of the target task model after the parameters are shared comprises:
inputting the parameters of the sample electrocardio-signals into the target task model after the parameters are shared, inputting the output of the target task model and the target abnormal labels into a preset fourth loss function, to obtain a fourth loss value, and adjusting the parameters of the target task model with a goal of minimizing the fourth loss value.

In an optional embodiment, the third loss function and the fourth loss function are both cross-entropy loss functions.

In an optional embodiment, the step of training the multi-task model based on the multi-task learning mechanism comprises:
performing multiple rounds of iterative training to the multi-task model based on the multi-task learning mechanism; wherein, each round of iterative training includes: adjusting parameters of each of the related task models, and the step of adjusting the parameters of the target task model according to the parameters of the at least one related task model.

In an optional embodiment, the step of adjusting parameters of each of the related task models comprises:
performing multiple rounds of iterative adjustment respectively on the parameters of each of the related task models, until each of the related task models satisfies a corresponding training stop condition, and determining related task models after trained as different related abnormality identification models; and
the step of adjusting the parameters of the target task model according to the parameters of the at least one related task model comprises:
adjusting the parameters of the target task model according to the parameters of the at least one related abnormality identification model.

The present disclosure provides a signal recognition method, comprises:
acquiring target electrocardio-signals; and
inputting the target electrocardio-signals into a target-abnormality-recognition model, to obtain target abnormality identification results, the target abnormality identification results are configured to indicate whether the target electrocardio-signals have a target abnormality; wherein, the target-abnormality-recognition model is obtained by training with the model training method according to any one of the above embodiments.

The present disclosure provides a model training apparatus, comprising:
a sample acquiring module configured for, acquiring a training sample set, the training sample set includes sample electrocardio-signals and abnormal labels of the sample electrocardio-signals, and the abnormal labels include target abnormal labels and at least one related abnormal label;
a model training module configured for, inputting the sample electrocardio-signals into a multi-task model, and training the multi-task model based on a multi-task learning mechanism according to output of the multi-task model and the abnormal labels; wherein, the multi-task model includes a target task model and at least one related task model, target output of the target task model is target abnormality labels of the inputted sample electrocardio-signals, and target output of the related task model is the related abnormal labels of the inputted sample electrocardio-signals; and
a model determining module configured for, determining the target task model after trained as a target-abnormality-recognition model, and the target-abnormality-recognition model is configured for recognizing a target abnormality in the electrocardio-signals inputted into the target-abnormality-recognition model.

The present disclosure provides a signal recognition apparatus, comprising:
a signal acquiring module configured for acquiring target electrocardio-signals; and
an abnormality identification module configured for, inputting the target electrocardio-signals into a target-abnormality-recognition model, to obtain target abnormality identification results, the target abnormality identification results are configured to indicate whether the target electrocardio-signals have a target abnormality; wherein, the target-abnormality-recognition model is obtained by training by the model training method according to any one of the above embodiments.

The present disclosure provides a computing and processing device, comprises:
a memory, wherein the memory stores a computer-readable code;
one or more processors, when the computer-readable code is executed by one or more processors, the computing and processing device executes the method according to any one of the above embodiments.

The present disclosure provides a computer program comprising a computer-readable code, when the computer readable code is executed on the computing and processing device, it causes the computing and processing device to execute the method according to any one of the above embodiments.

The present disclosure provides a computer-readable medium, wherein the computer-readable medium stores the method according to any one of the above embodiments.

The above description is merely an overview of the technical solutions of the present disclosure. In order to more apparently understand the technical means of the present disclosure to implement in accordance with the contents of specification, and to more readily understand above and other objectives, features and advantages of the present disclosure, specific embodiments of the present disclosure are provided hereinafter.

### BRIEF DESCRIPTION OF THE DRAWINGS

In order to more clearly illustrate the technical solutions of the embodiments of the present disclosure or the prior art, the figures that are required to describe the embodiments or the prior art will be briefly introduced below. Apparently, the figures that are described below are embodiments of the present disclosure, and a person skilled in the art can obtain other figures according to these figures without paying creative work. It should be noted that the ratios in the drawings are merely illustrative and do not represent actual ratios.

FIG. 1 schematically illustrates a flow chart of a model training method;

FIG. 2 schematically illustrates a flow chart of a signal recognition method;

FIG. 3 schematically illustrates another type of flow chart showing the process of training to obtain a target-abnormality-recognition model;

FIG. 4 schematically illustrates a schematic diagram of a soft parameter-sharing multi-task model;

FIG. 5 schematically illustrates a dual-channel neural network model;

FIG. 6 schematically illustrates a schematic diagram of a hard parameter-sharing multi-task model;

FIG. 7 schematically illustrates a block diagram of a model training apparatus;

FIG. 8 schematically illustrates a block diagram of a signal recognition apparatus;

FIG. 9 schematically illustrates a block diagram of a computing and processing device for executing methods according to the present disclosure; and

FIG. 10 schematically illustrates a memory unit for holding or carrying program code executing the method according to the present disclosure.

### DETAILED DESCRIPTION OF THE EMBODIMENTS

In order to make the objects, the technical solutions, and the advantages of the embodiments of the present disclosure clearer, the embodiments of the present disclosure will be clearly and completely described below with reference to the drawings of the embodiments of the present disclosure. Apparently, the described embodiments are merely certain embodiments of the present disclosure, rather than all of the embodiments. All of the other embodiments that a person skilled in the art obtains on the basis of the embodiments of the present disclosure without paying creative work fall within the protection scope of the present disclosure.

FIG. 1 schematically shows a flow chart of a model training method. As shown in FIG. 1, the method may include the following steps.

Step 11: acquiring a training sample set, the training sample set includes sample electrocardio-signals and abnormal labels of the sample electrocardio-signals, and the abnormal labels include target abnormal labels and at least one related abnormal label;

The execution subject of the embodiment may be a computer device, and the computer device includes a model training device, and the model training method provided in the embodiment is executed by the model training device. The computer device may be, for example, a smart phone, a tablet computer, a personal computer, or the like, which is not limited in the embodiment.

The execution subject of the embodiment may obtain the training sample set in various ways. For example, the execution subject may obtain the sample electrocardio-signals stored in another server (such as a database server) for storing data through a wired connection way or a wireless connection way. For another example, the execution subject may acquire the sample electrocardio-signals collected by a signal acquisition device such as an electrocardiogramd store these sample electrocardio-signals locally, thereby generating the training sample set.

Abnormalities of the sample electrocardio-signals may include: at least one of the anomalies, such as atrial premature beats, ventricular premature beats, supraventricular tachycardia, ventricular tachycardia, atrial flutter, atrial fibrillation, ventricular flutter, ventricular fibrillation, left bundle branch block, right bundle branch block, atrial sexual escape, ventricular escape, tachycardia, bradycardia, atrioventricular block, ST-segment elevation, ST-segment depression, Brugada wave abnormality, giant R-wave ST-segment elevation and camouflaged bundle branch block. Among them, the sample data of the atrial flutter, escape, the ST-segment elevation, the ST-segment depression, the Brugada wave abnormality, the giant R-wave ST-segment elevation, and camouflaged bundle branch block are relatively few.

The characteristics of the electrocardio-signals may include waveform, wave peak, wave amplitude, frequency, amplitude, time, and the like. Some abnormal Electrocardio-signals have commonalities or similarities in characteristics, which are reflected in the same features of some waveforms or the same upper and lower frequency thresholds. For example: (1) The lower limit value of the heart rate threshold for abnormalities such as supraventricular tachycardia, paroxysmal tachycardia, atrial fibrillation, atrial flutter, and atrial tachycardia is 100 beats/min, while the upper limit value of the heart rate threshold for abnormalities such as sinus tachycardia, atrioventricular conduction block, sinoatrial conduction block, and bundle branch conduction block is 60 beats/min; (2) when the ventricular rate is high, it has similar characteristics to the abnormal electrocardio-signals such as supraventricular tachycardia and sinus tachycardia; (3) masquerade bundle branch block, left bundle branch block in limb lead ECG images, and right bundle branch block in precordial lead ECG images; (4) the relatively rare Brugada wave abnormality was extracted in North America in 1991. This abnormality presents the ECG features of the right bundle branch block with ST-segment elevation in the right chest leads; (5) the abnormality of giant R-wave ST-segment elevation first proposed by Wimalarlna in 1993 has the waveform characteristics of QRS fused with elevated ST-segment, and upright T wave. (6) the J wave first discovered in 1938, also known as the “Osborn” wave, is very similar in shape to a part of the QRS complex and the second R wave.

The characteristics of these abnormal Electrocardio-signals have commonalities, so these abnormalities are correlated, meet the conditions of multi-task learning, and can transfer knowledge between tasks.

In the specific implementation, there is a correlation between the target abnormality indicated by the target abnormal labels and the related abnormality indicated by the related abnormal labels. For example, the target abnormality is atrial flutter, and the related abnormality is atrial fibrillation. When the number of related abnormalities is plural, each of the related abnormalities has a correlation with the target abnormality. The number of the sample electrocardio-signals with any of the related abnormalities in the training sample set may be greater than the number of the sample electrocardio-signals with the target abnormality.

In the embodiment, the training sample set may include multiple sample electrocardio-signals, and it is assumed that these sample electrocardio-signals involve M types of abnormalities, and the M types of abnormalities include first abnormality, second abnormality . . . and M-th abnormality. M is greater than or equal to 2. Assuming that any one of the first abnormality is the target abnormality, and any abnormality of the second abnormality, the third abnormality . . . and the M-th abnormality is related to the first abnormality and is different from the first abnormality. Therefore, any abnormality of the second abnormality, the third abnormality . . . and the M-th abnormality may be the related abnormality.

When M=2, the number of the related abnormalities is one, that is the second abnormality; when M≥3, the number of the related abnormalities is multiple, the multiple related abnormalities are, respectively, the second abnormality, the third abnormality . . . and the M-th abnormality.

In the specific implementation, the abnormal labels of each sample electrocardio-signals can be a vector of M dimension. For example, the abnormal label of a certain sample electrocardio-signals is [1, 0, 0, 1, 1, . . . , 1], 1 in the abnormal labels represents that the sample electrocardio-signals has a corresponding abnormality, 0 represents that there is no corresponding abnormality, and the above abnormal labels represent that the sample electrocardio-signals has the first abnormality, the fourth abnormality, the fifth abnormality, . . . and the M-th abnormality.

In the specific implementation, the sample electrocardio-signals in the training sample set can be classified according to the type of abnormalities. After classification, the sample electrocardio-signals corresponding to abnormality i can contain two groups: positive samples with i-th abnormality and negative samples without i-th abnormality. Wherein, i can be greater than or equal to 1 and less than or equal to M. The number of the positive samples and the number of negative samples can be equal or relatively close. In the specific implementation, the ratio of the positive samples to the negative samples can be adjusted according to actual needs, which is not limited in the embodiment.

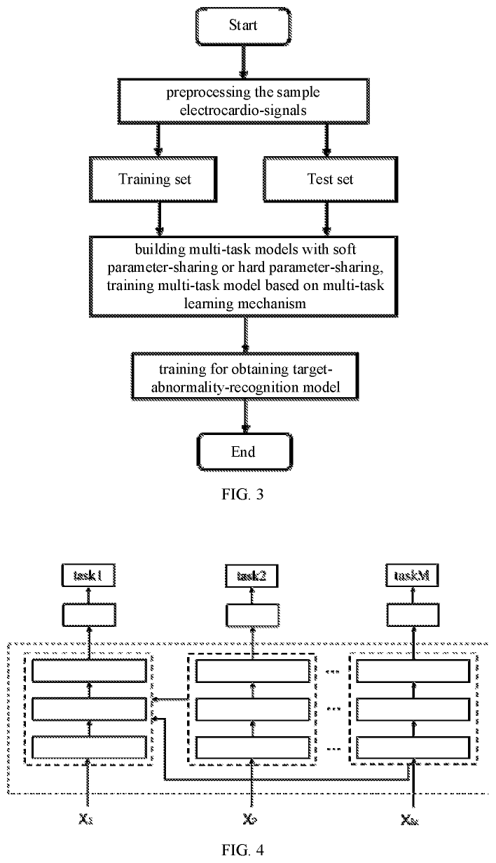

In the specific implementation, it is also possible to preprocess the sample electrocardio-signals before the step S12, as shown in the FIG. 3, to remove noise interference. Specifically, a power interference with 50 Hz in the sample electrocardio-signals can be removed with a band-pass filter; an electromyographic interference with 10-300 Hz in the sample electrocardio-signals can be removed with a low-pass filter; a baseline drift in the sample electrocardio-signals can be removed with a high-pass filter; and so on.

In the specific implementation, the sample electrocardio-signals in the training sample set can also be divided into a training set and a test set according to a certain ratio, such as 4:1, as shown in the FIG. 3, which is not limited in the embodiment.

Step S12: inputting the sample electrocardio-signals into a multi-task model, and training the multi-task model based on a multi-task learning mechanism according to output of the multi-task model and the abnormal labels; wherein, the multi-task model includes a target task model and at least one related task model, target output of the target task model is target abnormality labels of the inputted sample electrocardio-signals, and target output of the related task model is the related abnormal labels of the inputted sample electrocardio-signals;

Step S13: determining the target task model after trained as a target-abnormality-recognition model, and the target-abnormality-recognition model is configured for recognizing a target abnormality in the electrocardio-signals inputted into the target-abnormality-recognition model.

Wherein, the target task model and each of the related task models may be neural network model with the same network structure, for example, a Convolutional Neural Networks (CNN) model or a Recurrent Neural Network (RNN) model. Specifically, the target task model and each of the related task models may use a Long Short-Term Memory (LSTM) in the RNN model. Certainly, the target task model and each of the related task models may also be models with different network structures, which are not limited in the embodiment.

Multi-task learning (MTL) is an important machine learning method that aims to improve the generalization ability of the main task by using related tasks. In the process of multi-task learning, the relationship between tasks is captured by constraining the relationship between the model parameters of each of the tasks, so that the knowledges learned from the related tasks with more training data is transferred to the main task with less training data. The multi-task learning imposes some certain constraints on the main task, that is, the parameters of the main task model are constrained by the parameters of the related task model during the optimization process, so that when all tasks meet the convergence conditions, the main task model is equivalent to integrate all of the knowledges learned from the related task models, so as to improve the generalization ability of the main task.

In the embodiment, since the target abnormality is correlated with each of the related abnormalities, that is, the electrocardio-signals with the target abnormality and the electrocardio-signals with any kind of related abnormality have features in common. Therefore, the target-abnormality-recognition model for identifying the target abnormality and the related abnormality identification model for identifying the related abnormality can be obtained by training with a multi-task learning mechanism.

Since the number of the sample electrocardio-signals with the target abnormality is less than the number of the sample electrocardio-signals with any one of the related abnormalities, in the process of training the target-abnormality-recognition model by using the multi-task learning mechanism, the task of training the target-abnormality-recognition model is the main task, such as the task 1 shown in the FIG. 4 and FIG. 6. The main task is used to train the target task model in the multi-task model, the target task model after trained is a target-abnormality-recognition model, and the target-abnormality-recognition model is used to recognize whether the electrocardio-signals inputted the target-abnormality-recognition model have target abnormalities, such as the first abnormalities.

The task of training the related abnormality identification model is the related task, the related task is used to train the related task model, and the trained related task model is the related abnormality identification model. In the multi task learning, the number of the related task and the related task model is at least one, since the number of the related abnormality is M−1, correspondingly, the number of the related task and the related task model can both be M−1. The M−1 related tasks are, respectively, task 2 . . . task M, as shown in the FIG. 4 and FIG. 6.

Specifically, if the number of related tasks is one, the related task is used to train a related task model; if the number of the related tasks is multiple, each of the related tasks is used to train a related task model, and each of the related tasks is used to train different related task models, each of the related task models after trained can be determined as a different related abnormality identification model. Each of the related abnormality identification models can be used to identify different related abnormalities.

In the specific implementation, the multi-task learning on the target task model and at least one related task model is performed, for example, two approaches are used, namely a hard parameter-sharing and a soft parameter-sharing. Among them, the hard parameter-sharing is to share the hidden layer of the network between multiple task models, that is, the related task model and the target task model. The parameters of the hidden layer in the multiple task models are the same, and the network output layer of each of the task models is different, so as to perform different tasks. The soft parameter-sharing refers to that each of the tasks has its own model and parameters, but the parameters of the main task model, i.e. the target task model, are constrained by the parameters of the related task model, to encourage parameters similarity between the target task model and the related task model. The detailed process of training the multi-task model based on the multi-task learning mechanism will be introduced in the subsequent embodiments.

In the embodiment, in the process of training the target task model and the related task model by using the multi-task learning mechanism, the parameters of the target task model are constrained by the parameters of the related task model, and the target task model is obtained by training based on the parameters of the related task model, so that the knowledge (i.e. parameters) learned from the related task model with more training data can be transferred to the target task model with less training data. Due to the number of the sample electrocardio-signals with the related abnormality are huge, and the target abnormality has correlation with the related abnormality, the generalization ability and the classification and recognition effect of the target-abnormality-recognition model trained by the multi-task learning mechanism are improved.

The model training method provided in the embodiment uses the multi-task learning mechanism to train the target task model and the related task model in the multi-task model, so that the target task model with less training data integrates the knowledges (i.e. parameters) learned from the related task model with more training data, the target task model after trained is the target-abnormality-recognition model, so it can improve the generalization ability and classification performance of the target-abnormality-recognition model, and it can effectively solve the problems of the poor classification and the recognition effect of the target-abnormality-recognition model caused by the insufficient sample data with target abnormality.

In an alternative implementation, the step of training multi-task model based on the multi-task learning mechanism in the step S12 may specifically include: firstly, adjusting parameters of each of the related task models, and adjusting the parameters of the target task model according to the parameters of the at least one related task model.

The process of training the multi-task model using the soft parameter-sharing method in the step S12 is described as following.

Referring to FIG. 4, a schematic diagram of training a multi-task model using the soft parameter-sharing method is shown. The step of adjusting the parameters of the target task model according to the parameters of the at least one related task model in the step S12 may include: determining a regularized-loss item according to the parameters of the target task model and the parameters of the at least one related task model, and the regularized-loss item is configured to make the parameters of the target task model and the parameters of the at least one related task model similar; determining a first loss value according to the regularized-loss item, and adjusting the parameters of the target task model with a goal of minimizing the first loss value.

In the specific implementation, the regularized-loss item can be determined according to the following formula:

R(θ₁,θ₂ , . . . , θ_{M})=λ(|θ₁−θ₂|²+. . . +|θ₁−θ_{M}|²)

Wherein, R (θ₁,θ₂, . . . , θ_{M}) refers to the regularized-loss item; M refers to total number of the target task model and related task model in the multi-task model, that is the number of task in the multi task training; θ₁ refers to the parameters of the target task model; θ₂, . . . , θ_{M} refer to respectively the parameters of each of the related task models; λ indicates preset parameters, λ is a hyperparameter, and its value can be set according to the sample distribution and the experience. By determining the regularized-loss item, the parameters of the target task model can be promoted to be similar to the parameters of the related task model.

In this implementation, the parameters of the target task model are constrained by adding a regularized-loss item to the loss function of the target task model. And since the regularized-loss item is determined and obtained according to the parameters of the related task model, the parameters of the target task model can be constrained by the related task model, so that the knowledges learned by the related task model with a large number of the sample electrocardio-signals can be transferred to the target task model, so as to improve the classification and recognition performance of the target task model.

In this implementation, the step of adjusting the parameters of each of the related task models in the step 12 may specifically include:
inputting the sample electrocardio-signals into a first related task model, and inputting the output of the first related task model and first related abnormal labels into a preset second loss function, to obtain a second loss value, adjusting the parameters of the first related task model with a goal of minimizing the second loss value, wherein, the first related task model is any one of the at least one related task model, and the first related abnormal label is the any one of the at least one related abnormal labels. The first related abnormal label is any one of the at least one related abnormal labels of the sample electrocardio-signals inputted into the first related task model.

In this implementation, before the step of determining the first loss value according to the regularized-loss item in the step S12, further comprises: inputting the sample electrocardio-signals into the target task model, inputting the output of the target task model and the target abnormal labels into a preset experience loss function, to obtain an experience loss item; wherein, the experience loss function may be a cross-entropy loss function.

Furthermore, the step of determining a first loss value according to the regularized-loss item, may include: calculating the sum of the experience loss item and the regularized-loss item, to obtain the first loss value. After that, the target task model can be trained with the goal of minimizing the first loss value.

In the specific implementation, the Convolutional Neural Networks can be used to establish the target task model C₁ and M−1 related task models C₂ . . . C_{M}, which have the same network structure.

Wherein, the target task model C₁ can be represented by the following formula: Y₁=f (θ₁,X₁), wherein, θ₁ refers to the parameters of the target task model C₁, X₁ refers to the input of the target task model C₁, Y₁ refers to the output of the target task model C₁.

Any one of the related task models (i.e. the first related abnormality model mentioned above) Cᵢ can be represented by the following formula: Yᵢ=f (θᵢ,Xᵢ), wherein, θᵢ refers to the parameters of the related task model Cᵢ, Xᵢ refers to the input of the related task model Cᵢ, Yᵢ refers to the output of the related task model Cᵢ, wherein, 2≤i≤M.

The first loss value T1 of the target task model C₁ can be the sum of the experience loss item E and the regularized-loss item R (θ₁,θ₂ , . . . , θ_{M}), that is T1=E+R (θ₁,θ₂ , . . . , θ_{M}). Wherein, the experience loss item E can be calculated by using the cross-entropy loss functions.

The second loss value T2 of any one of the related task models C₁ can be a cross-entropy loss function.

Wherein, the calculating formula of the cross-entropy loss functions is represented as following:

$$E = - \frac{1}{N} {\sum }_{n} {\sum }_{k} {t}_{nk} log {y}_{nk} ,$$

In the above formula, N represents the number of sample electrocardio-signals in the training set, the summation of the inner layer is the loss function of a single sample electrocardio-signals, and the summation of the outer layer is the loss function of all sample electrocardio-signals, then the summation result is divided by N, to get the average loss function. tₙₖ is the sign function, if the actual category of the n-th sample electrocardio-signals is k, then the value is 1, otherwise the value is 0. yₙₖ represents the output of the network model, that is, the probability that the n-th sample electrocardio-signals belongs to the abnormal type k.

By the calculating the experience loss item in the first loss value T1, there are only two categories k in the cross-entropy loss functions, that is, the category with target abnormality and the category without target abnormality. tₙₖ represents the target abnormal labels of the n-th sample electrocardio-signals. When the n-th sample electrocardio-signals has target abnormality, tₙₖ takes the value 1; otherwise, it takes the value 0. yₙₖ represents the output of target task model C₁, that is, the probability that the n-th sample electrocardio-signals with target abnormality.

By the calculating the second loss value T2 of the first related abnormality model, there are only two categories k in the cross-entropy loss functions, that is, the category with first related abnormality and the category without first related abnormality. tₙₖ represents the first related abnormal labels of the n-th sample electrocardio-signals. When the n-th sample electrocardio-signals has the first related abnormality, tₙₖ takes the value 1; otherwise, it takes the value 0. yₙₖ represents the output of the first related task model Cᵢ that is, the probability that the n-th sample electrocardio-signals with the first related abnormality.

In the specific implementation, the process of training the neural network model mainly includes: forwarding propagation to calculate the actual output, backing propagation to calculate the error and optimize the loss function, updating and adjusting the model parameters by using the gradient descent algorithm layer by layer. The minimum error and an optimal loss function are obtained through multiple iterations of training, so as to complete the training of the neural network model.

In the embodiment, the step of training the multi-task model based on the multi-task learning mechanism can be implemented in multiple ways. In an alternative implementation, multiple rounds of iterative training can be performed to the multi-task model based on the multi-task learning mechanism; wherein, each round of the iterative training includes: the step of adjusting the parameters of each of the related task models, and adjusting the parameters of the target task model according to the parameters of the at least one related task model.

In this implementation, in each iteration cycle, for each of the at least one related task model, the sample electrocardio-signals can be firstly inputted into the related task model, and the output of the related task model and the corresponding related abnormal labels can be inputted into preset second loss function, to obtain the second loss value, and adjusting the parameters of the related task model with the goal of minimizing the second loss value. After adjusting the parameters of each related task model according to the above process, the sample electrocardio-signals can be inputted into the target task model, the output of the target task model and the target abnormal labels are inputted into the preset experience loss function, to obtain the experience loss item, at the same time, according to the parameters based on the target task model and the adjusted parameters of at least one (such as all) related task model, the regularized-loss item is determined. Then the sum of the experience loss item and the regularized-loss item is calculated, to obtain the first loss value; after that, the parameters in the target task model are adjusted with the goal of minimizing the first loss value, and thus an iteration cycle is completed. The multiple rounds of iterations are performed in sequence according to the above process, until the iteration stop conditions are met (such as the number of iterations reaching the set number, convergence, etc.), the training of the target task model and each related task model can be completed, and the target task model after trained is determined as the target-abnormality-recognition model, and each of the related task models after trained is determined as a different related abnormality identification model.

In another alternative implementation, the multiple rounds of iterative adjustment on the parameters of each of the related task models can be firstly performed, until each of the related task models satisfies the corresponding training stop condition, and each of the trained related task model is determined as a different related abnormality identification model; then the parameters of the target task model can be adjusted according to the parameters of at least one related abnormality identification model.

In this implementation, for each of at least one related task model, the sample electrocardio-signals can be firstly input to the related task model, the output of the first related task model and the first related abnormal labels can be input into the preset second loss function, and the second loss function can be obtained. Multiple rounds of iterative training are performed on the related task model with the goal of minimizing the second loss value, to obtain a related abnormality identification model.

After adjusting the training according to the above process to obtain each of the related abnormality identification models, the sample electrocardio-signals can be inputted into the target task model, the output of the target task model and the target abnormal labels can be inputted into the preset experience loss function, and the experience loss item can be obtained. At the same time, the regularized-loss item is determined based on the parameters of the target task model and the parameters of at least one (such as all) related abnormality identification model; then the sum of the experience loss item and the regularized-loss item is calculated to obtain the first loss value; After that, with a target of minimizing the first loss value, the target task model is trained, and the target task model after trained is determined as the target-abnormality-recognition model.

The process of training the multi-task model by the method of sharing hard parameters will be described in the step S12 below.

Referring to FIG. 6, a schematic diagram of training a multi-task model by the method of hard parameter-sharing is shown. As shown in FIG. 6, the target task model and the related task model share a common feature extraction layer, and the common feature extraction layer is used to extract the common features of the target abnormality and the related abnormality. The step of adjusting the parameters of the target task model according to the parameters of the at least one related task model in the step S12 may include: sharing parameters of the common feature extraction layer in the at least one related task model as parameters of the common feature extraction layer in the target task model, adjusting the parameters of the target task model after parameter sharing.

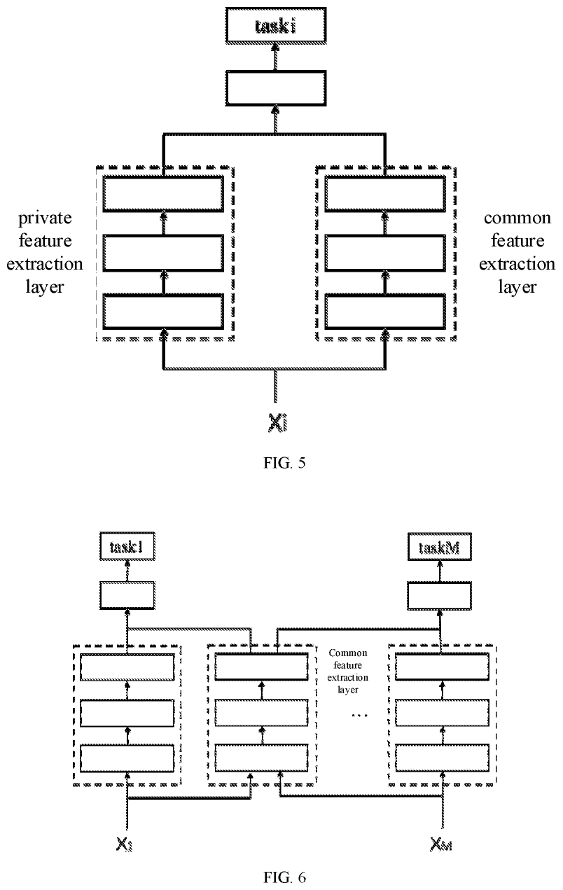

In this implementation, the target task model and each of the related task models are both dual-channel deep learning models as shown in FIG. 5. Either of the target task model and each of the at least one related task models includes a private feature extraction layer and a common feature extraction layer, wherein the private feature extraction layer is used to extract private features, and the common feature extraction layer is used to extract common features. By setting the private feature extraction layer, each of the task models can identify different abnormalities and achieve specificity; by setting the common feature extraction layer, the knowledges learned by the related task model can be transferred to the target task model, thereby improving the classification and recognition performance of the target task model.

In this implementation, the step S12 may include:

Firstly, inputting the sample electrocardio-signals into a second related task model, inputting output of the second related task model and the second related abnormal labels into the preset third loss function, to obtain the third loss value, and adjusting the parameters of the second related task model with the goal of minimizing the third loss value, the parameters of the second related task model include the parameters of the common feature extraction layer, wherein, the second related task model is any one of the at least one related task model, and the second related abnormal labels is any one of the at least one related abnormal label. The second related abnormal label is any one of the at least one related abnormal label of the sample electrocardio-signals inputted into the second related task model.

After that, the parameters of the common feature extraction layer in at least one related task model are shared as the parameters of the common feature extraction layer in the target task model.

Then, inputting the sample electrocardio-signals into the target task model after sharing parameters, inputting the output of the target task model and the target abnormal labels into the preset fourth loss function to obtain the fourth loss value, and adjusting the parameters of the target task model with the goal of minimizing the fourth loss value.

The parameter of private feature extraction layer in the target task model is θ₁, and the parameter of private feature extraction layer in at least one related task model is θ₂ . . . θ_{M}, the parameters of the common feature extraction layer of target task model and related task model are θ₀.

In the specific implementation, a convolutional neural network can be used to build a target task model and M−1 related task models with the same network structure.

Wherein, the target task model can be represented by the following formula: Y₁=f (θ₀, θ₁, X₁), wherein, X₁ represents the input of the target task model, Y₁ represents the output of the target task model.

Any one related task model (that is the second related task model mentioned above) can be represented by the following formula: Yᵢ=f (θ₀, θᵢ, Xᵢ), wherein, Xᵢ represents the input of the related task model, Yᵢ represents the output of the related task model.

Wherein, the third loss function and the fourth loss function can be cross-entropy loss functions. The calculating formula is shown as followings:

$$E = - \frac{1}{N} {\sum }_{n} {\sum }_{k} {t}_{nk} log {y}_{nk} ,$$

In the above formula, N represents the number of the sample electrocardio-signals in the training set, the summation of the inner layer is the loss function of a single sample electrocardio-signals, and the summation of the outer layer is the loss function of all sample electrocardio-signals, then the summation result is divided by N, to get the average loss function. tₙₖ is the sign function, if the actual category of the n-th sample electrocardio-signals is k, then the value is 1, otherwise the value is 0. yₙₖ represents the output of the network model, that is, the probability that the n-th sample electrocardio-signals belongs to the abnormal type k.

When calculating the third loss function of the second related task model, there are only two categories k in the cross-entropy loss function, that is, the category with the second related abnormality and the category without the second related abnormality. tₙₖ represents the second related abnormal labels of the n-th sample electrocardio-signals. When the n-th sample electrocardio-signals has the second related abnormality, tₙₖ takes the value 1; otherwise, it takes the value 0. yₙₖ represents the output of the second related task model, that is the probability of the n-th sample electrocardio-signals with the second related abnormality.

When calculating the fourth loss function, there are only two categories k in the cross-entropy loss function, that is, the category with the target abnormality and the category without the target abnormality. tₙₖ represents the target abnormal labels of the n-th sample electrocardio-signals. When the n-th sample electrocardio-signals has the target abnormality, tₙₖ takes the value 1; otherwise, it takes the value 0. yₙₖ represents the output of the target task model, that is, the probability of the n-th sample electrocardio-signals with the target abnormality.

In the specific implementation, the process of training the neural network model mainly includes: forwarding propagation to calculate the actual output, backing propagation to calculate the error and optimize the loss function, updating and adjusting the model parameters by using the gradient descent algorithm layer by layer. The minimum error and an optimal loss function are obtained through multiple iterations of training, so as to complete the training of the neural network model.

In the embodiment, the step of training the multi-task model based on the multi-task learning mechanism can be implemented in multiple ways. In an alternative implementation, multiple rounds of iterative training can be performed on the multi-task model based on the multi-task learning mechanism; wherein, each round of the iterative training includes: the step of adjusting the parameters of each of the related task models, and adjusting the parameters of the target task model according to the parameters of the at least one related task model.

In this implementation, in each iteration cycle, for each of at least one related task model, firstly, the sample electrocardio-signals are input into the related task model, the output of the related task model and the corresponding related abnormal labels are input into the preset third loss function to obtain the third loss value, and the parameters of the related task model are adjusted with the goal of minimizing the third loss value. After the parameters of related task model are adjusted according to the above process, the parameters of the common feature extraction layer in at least one related task model can be shared as the parameters of the common feature extraction layer in the target task model. After that, the sample electrocardio-signals are input into the target task model after parameters sharing, and the output of the target task model and the target abnormal labels are input into the preset fourth loss function to obtain the fourth loss value. Then, the parameters in the target task model can be adjusted with the goal of minimizing the fourth loss value, and a round of iteration cycle is completed. According to the above process, the multiple rounds of iterations are performed in sequence until the iteration stop conditions (i.e. the number of iterations reaching the set number, etc.) are met, and the training of the target task model and the related task models can be completed. The target task model after trained is determined as the target-abnormality-recognition model, and the related task models after trained are determined as a different related abnormality identification model.

In another alternative implementation, the parameters of the related task models are adjusted iteratively for multiple rounds, until the related task model satisfies the corresponding training stop condition. The related task model is determined as a different related abnormality identification model; afterwards, the parameters of the target task model can be adjusted according to the parameters of at least one related abnormality identification model.

In this implementation, for each of at least one related task model, firstly, the sample electrocardio-signals can be inputted into the related task model, inputting the output of the related task model and the corresponding related abnormal labels into the preset third loss function to obtain the third loss value, and with the goal of minimizing the third loss value, multiple rounds of iterative training are performed on the related task model to obtain a related abnormality identification model. After the above-mentioned process of adjustment and training to obtain the related abnormality recognition models, the parameters of the common feature extraction layer in at least one related abnormality identification model may be shared as the parameters of the common feature extraction layer in the target task model. After that, the sample electrocardio-signals can be inputted into the target task model after parameters sharing, and the output of the target task model and the target abnormal labels can be inputted into the preset fourth loss function to obtain the fourth loss value. Then, the target task model can be trained with the goal of minimizing the fourth loss value, and the target task model after trained is determined as the target-abnormality-recognition model.

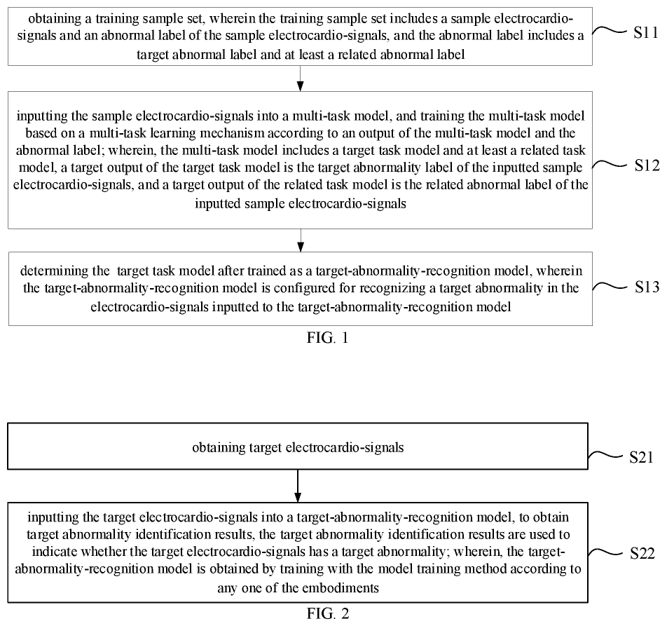

FIG. 2 schematically shows a flow chart of a signal recognition method. As shown in FIG. 2, the method may include the following steps.

Step S21: acquiring target electrocardio-signals.

In the embodiment, this step may specifically include the following steps: firstly, acquiring the original electrocardio-signals; then pre-processing the original electrocardio-signals to obtain target electrocardio-signals.

The execution subject in the embodiment may be a computer device, and the computer device includes a signal identification device, and the signal identification method provided in the embodiment is executed by the signal identification device. The computer device may be, for example, a smart phone, a tablet computer, a personal computer, or the like, which is not limited in the embodiment.

The execution subject of the embodiment can obtain the original electrocardio-signals in various ways. For example, the execution subject can obtain the original electrocardio-signals collected by signal acquisition equipment such as an electrocardiogramd then preprocessing the obtained original electrocardio-signals to obtain the target electrocardio-signals.

Through the preprocessing process, the format of the target electrocardio-signals can be the same as the format of the sample electrocardio-signals that inputted by training the target-abnormality-recognition model. In an alternative implementation, the step of preprocessing the original electrocardio-signals may include at least one of the following steps: using a band-pass filter to remove the power interference in the original electrocardio-signals; using a low-pass filter to remove the power interference in the original electrocardio-signals electromyographic interference; and, using a high-pass filter to remove baseline drift in the original electrocardio-signals.

Specifically, a band-pass filter can be used to remove 50 Hz power interference; a low-pass filter can be used to remove 10-300 Hz electromyographic interference; a high-pass filter can be used to remove baseline drift. By preprocessing the original electrocardio-signals, the noise interference in the original electrocardio-signals can be removed, and the accuracy of classification and recognition can be improved.

Step S22: inputting the target electrocardio-signals into a target-abnormality-recognition model, to obtain target abnormality identification results, the target abnormality identification results are used to indicate whether the target electrocardio-signals have a target abnormality; wherein, the target-abnormality-recognition model is obtained by training with the model training method according to any one of the embodiments.

In the specific implementation, the target electrocardio-signals can be inputted into the target-abnormality-recognition model, and target abnormality identification results can be outputted. According to the outputted target abnormality identification results, it can be determined whether the target electrocardio-signals have the target abnormality. The target abnormality identification results may include, for example, a probability that the target electrocardio-signals includes the target abnormality and a probability that the target electrocardio-signals do not includes the target abnormality, which are not limited in the embodiment.

Wherein, the target-abnormality-recognition model may be pre-trained, or may be obtained by training in the process of the signal recognition, which is not limited in the embodiment.

In the signal recognition method provided in the embodiment, since the target-abnormality-recognition model is obtained by training with the related abnormality identification model multi-task based on the learning mechanism, the target-abnormality-recognition model with less training data integrates the knowledge (i.e. parameters) learned by the related abnormality identification model with more training data, thereby improving the generalization ability and the classification performance of the target-abnormality-recognition model and improving the accuracy of the target abnormality identification.

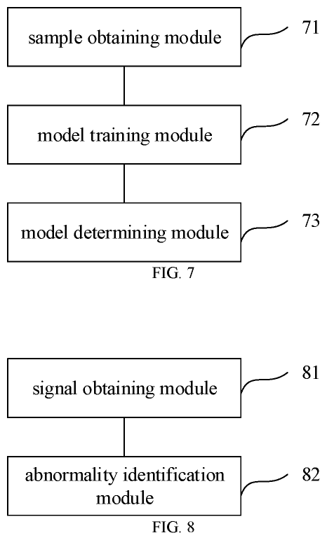

FIG. 7 schematically shows a block diagram of a model training apparatus. Referring to FIG. 7, may include:
a sample acquiring module 71 configured for acquiring a training sample set, the training sample set includes sample electrocardio-signals and abnormal labels of the sample electrocardio-signals, and the abnormal labels include target abnormal labels and at least one related abnormal label.
a model training module 72 configured for inputting the sample electrocardio-signals into a multi-task model, and training the multi-task model based on a multi-task learning mechanism according to output of the multi-task model and the abnormal labels; wherein, the multi-task model includes a target task model and at least one related task model, target output of the target task model is target abnormality labels of the inputted sample electrocardio-signals, and target output of the related task model is the related abnormal labels of the inputted sample electrocardio-signals.
a model determining module 73 configured for determining the target task model after trained as a target-abnormality-recognition model, and the target-abnormality-recognition model is configured for recognizing a target abnormality in the electrocardio-signals inputted into the target-abnormality-recognition model.

Regarding the apparatus in the above embodiment, the specific ways in which the various modules perform operations have been described in detail in the examples related to the model training method, for example, it is implemented by means of software, hardware, firmware, etc., which will not be described and illustrates in detail herein.

FIG. 8 schematically shows a block diagram of a signal recognition apparatus.

Referring to FIG. 8, it can include:
a signal acquiring module 81 configured for acquiring target electrocardio-signals.
an abnormality identification module 82 configured for, inputting the target electrocardio-signals into a target-abnormality-recognition model, to obtain target abnormality identification results, the target abnormality identification results are used to indicate whether the target electrocardio-signals have a target abnormality; wherein, the target-abnormality-recognition model is obtained by training by the model training method according to any one of the embodiments.

Regarding the apparatus in the above embodiment, the specific ways in which the various modules perform operations have been described in detail in the examples related to the model training method, for example, it is implemented by means of software, hardware, firmware, etc., which will not be described and illustrates in detail herein.

Apparatus embodiments set forth above are merely exemplary, wherein units described as detached parts may be or not be detachable physically; parts displayed as units may be or not be physical units, i.e., either located at the same place, or distributed on a plurality of network units. Modules may be selected in part or in whole according to actual needs to achieve objectives of the solution of the embodiment. Those of ordinary skill in the art may comprehend and implement the embodiment without contributing creative effort.

Each of devices according to the embodiments of the present disclosure can be implemented by hardware, or implemented by software modules operating on one or more processors, or implemented by the combination thereof. A person skilled in the art should understand that, in practice, a microprocessor or a digital signal processor (DSP) may be used to realize some or all of the functions of some or all of the parts in the electronic device according to the embodiments of the present disclosure. The present disclosure may further be implemented as equipment or device program (for example, computer program and computer program product) for executing some or all of the methods as described herein. Such program for implementing the present disclosure may be stored in the computer readable medium, or have a form of one or more signals. Such a signal may be downloaded from the Internet websites, or be provided on a carrier signal, or provided in any other form.

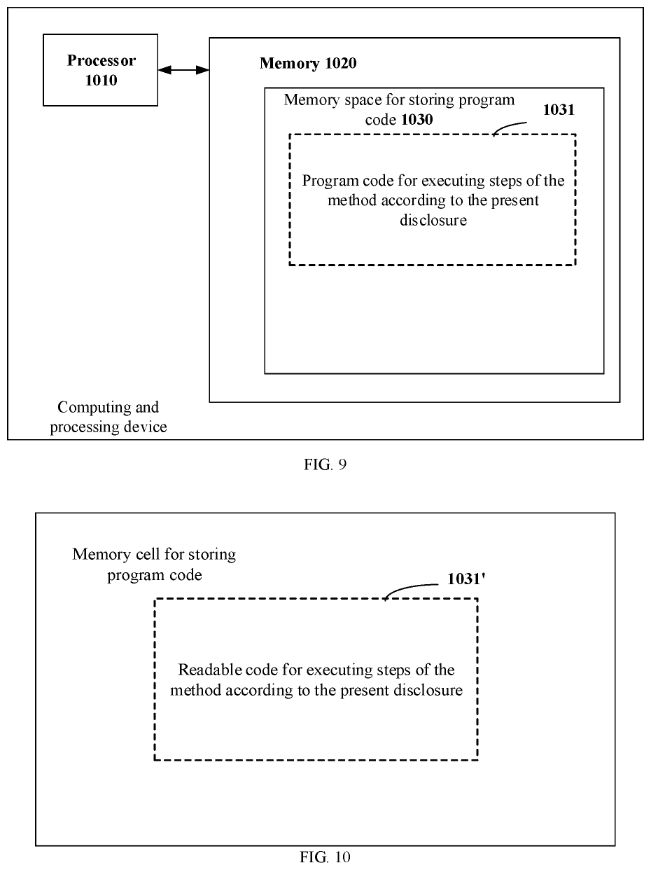

For example, FIG. 9 illustrates an electronic device that may implement the method according to the present disclosure. Traditionally, the electronic device comprises a processor 1010 and a computer program product or a computer readable medium in form of a memory 1020. The memory 1020 may be electronic memories such as flash memory, EEPROM (Electrically Erasable Programmable Read-Only Memory), EPROM, hard disk or ROM. The memory 1020 has a memory space 1030 for executing program codes 1031 of any steps in the above methods. For example, the memory space 1030 for program codes may comprise respective program codes 1031 for implementing the respective steps in the method as mentioned above. These program codes may be read from and/or be written into one or more computer program products. These computer program products include program code carriers such as hard disk, compact disk (CD), memory card or floppy disk. These computer program products are usually the portable or stable memory cells as shown in reference FIG. 10. The memory cells may be provided with memory sections, memory spaces, etc., similar to the memory 1020 of the electronic device as shown in FIG. 9. The program codes may be compressed for example in an appropriate form. Usually, the memory cell includes computer readable codes 1031′ which can be read for example by processors 1010. When these codes are operated on the electronic device, the electronic device may be caused to execute respective steps in the method as described above.

The embodiments in the specification of the present disclosure are described in a progressive manner. Each embodiment is focused on difference from other embodiments. And cross reference is available for identical or similar parts among different embodiments.

Finally, it should be explained that a relational term (such as a first or a second . . . ) is merely intended to separate one entity or operation from another entity or operation instead of acquiring or hinting any practical relation or sequence exists among these entities or operations. Furthermore, terms such as “comprise”, “include” or other variants thereof are intended to cover a non-exclusive “comprise” so that a process, a method, a merchandise or a device comprising a series of elements not only includes these elements, but also includes other elements not listed explicitly, or also includes inherent elements of the process, the method, the merchandise or the device. In the case of no more restrictions, elements restricted by a sentence “include a . . . ” do not exclude the fact that additional identical elements may exist in a process, a method, a merchandise or a device of these elements.

A model training method, signal recognition method, apparatus, computing and processing device, computer program, and computer-readable medium provided by the present disclosure have been introduced in detail above. The specific examples are used in this paper to demonstrate the principles and implementations of the present disclosure. For elaboration, the description of the above embodiments is only configured to help understand the method of the present disclosure and its core idea; meanwhile, for those skilled in the art, according to the idea of the present disclosure, there will be changes in the specific implementation and application scope. In summary, the contents of this specification should not be construed as limiting the present disclosure.

It should be understood that although the various steps in the flow chart of the accompanying drawings are sequentially shown in the order indicated by the arrows, these steps are not necessarily executed in sequence of the order indicated by the arrows. Unless explicitly stated herein, the execution of these steps is not strictly limited to the order and may be performed in other orders. Moreover, at least one part of the steps in the flow charts of the accompanying drawings may include multiple sub-steps or multiple stages, and these sub-steps or stages are not necessarily executed at the same time, but may be executed at different times, and the execution sequence is also It does not have to be performed sequentially, but may be performed alternately or alternately with other steps or at least one portion of sub-steps or stages of other steps.

Other embodiments of the present disclosure will readily occur to those skilled in the art upon consideration of the specification and practice of the invention disclosed herein. This disclosure is intended to cover any variations, uses, or adaptations of this disclosure that follow the general principles of this disclosure and include common general knowledge or techniques in the technical field not disclosed by this disclosure. The specification and examples are to be regarded as exemplary only, with the true scope and spirit of the disclosure being indicated by the following claims.

It is to be understood that the present disclosure is not limited to the precise structures described above and illustrated in the accompanying drawings, and the various modifications and changes may be made without departing from the scope thereof. The scope of the present disclosure is limited only by the appended claims.

The “one embodiment”, “an embodiment” or “one or more embodiments” as used herein means that particular features, structures or characteristics described with reference to an embodiment are included in at least one embodiment of the present application. Moreover, it should be noted that here an example using the wording “in an embodiment” does not necessarily refer to the same one embodiment.

The description provided herein describes many concrete details. However, it can be understood that the embodiments of the present application may be implemented without those concrete details. In some of the embodiments, well-known processes, structures and techniques are not described in detail, so as not to affect the understanding of the description.

In the claims, any reference signs between parentheses should not be construed as limiting the claims. The word “comprise” does not exclude elements or steps that are not listed in the claims. The word “a” or “an” preceding an element does not exclude the existing of a plurality of such elements. The present application may be implemented by means of hardware comprising several different elements and by means of a properly programmed computer. In unit claims that list several devices, some of those devices may be embodied by the same item of hardware. The words first, second, third and so on do not denote any order. Those words may be interpreted as names.

Finally, it should be noted that the above embodiments are merely intended to explain the technical solutions of the present application, and not to limit them. Although the present application is explained in detail by referring to the above embodiments, a person skilled in the art should understand that he can still modify the technical solutions set forth by the above embodiments, or make equivalent substitutions to part of the technical features of them. However, those modifications or substitutions do not make the essence of the corresponding technical solutions depart from the spirit and scope of the technical solutions of the embodiments of the present application.

1. A model training method, comprising:
acquiring a training sample set, wherein the training sample set comprises sample electrocardio-signals and abnormal labels of the sample electrocardio-signals, and the abnormal labels comprise target abnormal labels and at least one related abnormal label;
inputting the sample electrocardio-signals into a multi-task model, and training the multi-task model based on a multi-task learning mechanism according to an output of the multi-task model and the abnormal labels; wherein, the multi-task model comprises a target task model and at least one related task model, a target output of the target task model is target abnormality labels of the inputted sample electrocardio-signals, and a target output of the related task model is the related abnormal labels of the inputted sample electrocardio-signals; and
determining the target task model after trained as a target-abnormality-recognition model, wherein the target-abnormality-recognition model is configured for recognizing a target abnormality in the electrocardio-signals inputted into the target-abnormality-recognition model.

2. The model training method according to claim 1, wherein, the step of training the multi-task model based on the multi-task learning mechanism comprises: adjusting the parameters of each of the related task models, and adjusting parameters of the target task model according to parameters of the at least one related task model.

3. The model training method according to claim 2, wherein, the step of adjusting the parameters of the target task model according to the parameters of the at least one related task model comprises:
determining a regularized-loss item according to the parameters of the target task model and the parameters of the at least one related task model, and the regularized-loss item is configured to make the parameters of the target task model and the parameters of the at least one related task model similar; and
determining a first loss value according to the regularized-loss item, and adjusting the parameters of the target task model with a goal of minimizing the first loss value.

4. The model training method according to claim 3, wherein, the step of determining the regularized-loss item according to the parameters of the target task model and the parameters of the at least one related task model comprises:
determining the regularized-loss item according to the following formula:
R(θ₁,θ₂ , . . . , θ_{M})=λ(θ₁−θ₂|²+. . . +|θ₁−θ_{M}|²)
wherein, R (θ₁,θ₂ , . . . , θ_{M}) refers to the regularized-loss item, M refers to a total number of the target task model and the related task model in the multi-task model, θ₁ refers to the parameters of the target task model, θ₂ , . . . , θ_{M} respectively represents the parameters of the related task models, and λ represents a preset parameter.

5. The model training method according to claim 3, wherein, the step of adjusting the parameters of each of the related task models comprises:
inputting the sample electrocardio-signals into a first related task model, and inputting the output of the first related task model and first related abnormal labels into a preset second loss function, to obtain a second loss value, adjusting the parameters of the first related task model with a goal of minimizing the second loss value, wherein, the first related task model is any one of the at least one related task model, and the first related abnormal labels is any one of the at least one related abnormal labels;
before the step of determining the first loss value according to the regularized-loss item, the method further comprises:
inputting the sample electrocardio-signals into the target task model, inputting the output of the target task model and the target abnormal labels into a preset experience loss function, to obtain an experience loss item; and
the step of determining a first loss value according to the regularized-loss item comprises:
calculating the sum of the experience loss item and the regularized-loss item, to obtain the first loss value.

6. The model training method according to claim 5, wherein, the second loss value and the experience loss function are both cross-entropy loss functions.

7. The model training method according to claim 2, wherein, the target task model and the related task model share a common feature extraction layer, and the common feature extraction layer is configured to extract the common features of the target abnormality and a related abnormality, the step of adjusting the parameters of the target task model according to the parameters of the at least one related task model comprises:
sharing parameters of the common feature extraction layer in the at least one related task model as parameters of the common feature extraction layer in the target task model, and adjusting the parameters of the target task model after the parameters are shared.

8. The model training method according to claim 7, wherein, the step of adjusting the parameters of each of the related task models comprises:
inputting the sample electrocardio-signals into a second related task model, and inputting an output of the second related task model and the second related abnormal labels into a preset third loss function, to obtain a third loss value, and adjusting parameters of the second related task model with a goal of minimizing the third loss value, wherein, the parameters of the second related task model comprise the parameters of the common feature extraction layer, the second related task model is any one of the at least one related task model, and the second related abnormal label is any one of the at least one related abnormal label; and
the step of adjusting the parameters of the target task model after the parameters are shared comprises:
inputting the sample electrocardio-signals into the target task model after the parameters are shared, inputting the output of the target task model and the target abnormal labels into a preset fourth loss function, to obtain a fourth loss value, and adjusting the parameters of the target task model with a goal of minimizing the fourth loss value.

9. The model training method according to claim 8, wherein, the third loss function and the fourth loss function are both cross-entropy loss functions.

10. The model training method according to claim 2, wherein, the step of training the multi-task model based on the multi-task learning mechanism comprises:
performing multiple rounds of iterative training to the multi-task model based on the multi-task learning mechanism; wherein, each round of iterative training comprises: adjusting parameters of each of the related task models, and the step of adjusting the parameters of the target task model according to the parameters of the at least one related task model.

11. The model training method according to claim 2, wherein, the step of adjusting parameters of each of the related task models comprises:
performing multiple rounds of iterative adjustment respectively on the parameters of each of the related task models, until each of the related task models satisfies a corresponding training stop condition, and determining related task models after trained as different related abnormality identification models; and
the step of adjusting the parameters of the target task model according to the parameters of the at least one related task model comprises:
adjusting the parameters of the target task model according to the parameters of the at least one related abnormality identification model.

12. A signal recognition method, comprising:
acquiring target electrocardio-signals; and
inputting the target electrocardio-signals into a target-abnormality-recognition model, to obtain target abnormality identification results, the target abnormality identification results are configured to indicate whether the target electrocardio-signals have a target abnormality; wherein, the target-abnormality-recognition model is obtained by training by the model training method according to claim 1.

13. (canceled)

14. (canceled)

15. A computing and processing device, comprising:
a memory, wherein the memory stores a computer-readable code;
one or more processors, when the computer-readable code is executed by one or more processors, the computing and processing device executes the method according to claim 1.

16. (canceled)

17. A computer-readable medium, wherein the computer-readable medium stores the method according to claim 1.

18. A computing and processing device, comprising:
a memory, wherein the memory stores a computer-readable code;
one or more processors, when the computer-readable code is executed by one or more processors, the computing and processing device executes the method according to claim 12.

19. The computing and processing device according to claim 15, wherein, the step of training the multi-task model based on the multi-task learning mechanism comprises: adjusting the parameters of each of the related task models, and adjusting parameters of the target task model according to parameters of the at least one related task model.

20. The computing and processing device according to claim 19, wherein the step of adjusting the parameters of the target task model according to the parameters of the at least one related task model comprises:
determining a regularized-loss item according to the parameters of the target task model and the parameters of the at least one related task model, and the regularized-loss item is configured to make the parameters of the target task model and the parameters of the at least one related task model similar; and
determining a first loss value according to the regularized-loss item, and adjusting the parameters of the target task model with a goal of minimizing the first loss value.

21. The computing and processing device according to claim 20, wherein, the step of adjusting the parameters of each of the related task models comprises:
inputting the sample electrocardio-signals into a first related task model, and inputting the output of the first related task model and first related abnormal labels into a preset second loss function, to obtain a second loss value, adjusting the parameters of the first related task model with a goal of minimizing the second loss value, wherein, the first related task model is any one of the at least one related task model, and the first related abnormal labels is any one of the at least one related abnormal labels;
before the step of determining the first loss value according to the regularized-loss item, the method further comprises:
inputting the sample electrocardio-signals into the target task model, inputting the output of the target task model and the target abnormal labels into a preset experience loss function, to obtain an experience loss item; and
the step of determining a first loss value according to the regularized-loss item comprises:
calculating the sum of the experience loss item and the regularized-loss item, to obtain the first loss value.

22. The computing and processing device according to claim 19, wherein the target task model and the related task model share a common feature extraction layer, and the common feature extraction layer is configured to extract the common features of the target abnormality and a related abnormality, the step of adjusting the parameters of the target task model according to the parameters of the at least one related task model comprises:
sharing parameters of the common feature extraction layer in the at least one related task model as parameters of the common feature extraction layer in the target task model, and adjusting the parameters of the target task model after the parameters are shared.

23. The computing and processing device according to claim 19, wherein the step of training the multi-task model based on the multi-task learning mechanism comprises:
performing multiple rounds of iterative training to the multi-task model based on the multi-task learning mechanism; wherein, each round of iterative training comprises: adjusting parameters of each of the related task models, and the step of adjusting the parameters of the target task model according to the parameters of the at least one related task model.
